(19) United States
(12) Patent Application Publication
(10) Pub. No.: US 2003/0053979 A1
(43) Pub. Date: Mar. 20, 2003

(54) Iridescent cosmetic composition and uses thereof

(76) Inventors: Laurence Arnaud-Sebillotte, L'Hay Les Roses (FR); Veronique Guillou, Antony (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 10/182,400

(22) PCT Filed: Jan. 14, 2002
(86) PCT No.: PCT/FR02/00131

(30) Foreign Application Priority Data
Jan. 18, 2001 (FR) 01/00687

(51) Int. Cl. A61K 7/021; A61K 7/075; A61K 7/08

(52) U.S. Cl. 424/70.31; 424/63

(57) ABSTRACT

The invention relates to a topically-applied iridescent composition comprising at least one surfactant selected from among alkypolyglycosides and an aqueous dispersion of polymer particles, said particles having an average size of between 50 and 300 nm. The inventive composition has a very attractive appearance owing to the iridescent color thereof and it is particularly suitable for use as a foaming composition, especially for cleansing the skin, including the scalp and/or hair. Said composition has very good foaming power.

The subject of the invention is an iridescent composition for topical application, comprising at least one alkyl polyglycoside and an aqueous dispersion of particles of polymer, the said particles having a mean size in numerical terms ranging from about 50 to 300 nm, and the uses of the said composition, in particular as foaming composition for cleansing of and/or removal of make-up from the skin including the scalp and/or the hair.

Care and/or cleansing compositions for the skin are generally in the form of transparent or white products depending on their constituent ingredients. To make them more attractive, it is possible to colour them by adding various colouring agents thereto. These colouring agents may be, for example, pigments such as lacquers, inorganic pigments or pearlescent pigments, or soluble colorants. However, these colouring agents are destabilized by light, hence a lack of homogeneity of the composition.

In particular, to obtain iridescent coloured effects, it is possible to use pearlescent pigments with a variety of colours. However, most often, the iridescent effect obtained with these pearlescent pigments is fairly weak, and, in addition, these pigments are difficult to disperse and to maintain in suspension, in particular in cleansing compositions which are often quite fluid. The incorporation of such pigments is therefore delicate and the reproducibility of the effect obtained is not certain.

It has therefore been sought to design products, in particular foaming cleansing products, having an iridescent effect without incorporating pearlescent pigments.

The Applicant has found, surprisingly, that the combination of polymers, in the form of particles in dispersion (in emulsion or in latex form), with particular surfactants, allows the production of products with an iridescent appearance, which are very attractive for consumers without having the technical difficulties of suspension of the particles and while giving products having a good foaming effect.

Indeed, compositions containing a colouring system, which is obtained from colloidal crystalline networks in the medium, giving an iridescent colour without the addition of pigments or colorants, are known from the document WO-A-00/47167. However, to obtain such an effect, the medium has to have a relatively low ionic strength and a conductance (the conductance is the product of the conductivity of the composition times the measuring cell constant) of less than 2.5 μΩ⁻¹, which limits the addition of ionic additives such as ionic surfactants and certain active agents. However, in cosmetic compositions and in particular in cleansing compositions, it is often necessary to introduce ionic compounds and in particular anionic surfactants into the medium. Moreover, according to this document, to obtain the iridescent effect, the medium must be very pure (see page 6, line 15 of WO-A-00/47167), which limits the possible applications a lot. In addition, it was not obvious that the incorporation of foaming surfactants into such compositions would be possible without affecting either the iridescent effect or the foaming power of the composition.

The need therefore remains for an iridescent, in particular foaming, composition which can retain this iridescent character regardless of the conductance of its constituent medium and regardless of its constituent compounds.

The Applicant has remedied the disadvantages of the prior art by using aqueous dispersions of particles of polymers, in which the mean size of the particles in numerical terms ranges from about 50 to 300 nm, and particular surfactants.

Accordingly, the subject of the present invention is an iridescent composition for topical application, comprising at least one surfactant chosen from alkyl polyglycosides, and monodisperse particles of polymer in aqueous dispersion, the said particles having a mean size in numerical terms ranging from 50 to 300 nm.

<<Mean size in numerical terms>> refers to the mean diameter in numerical terms of the preferably spherical particles of polymer of the dispersion, this size being the original size of the particles before they are mixed with other constituents. Indeed, the size of the particles may be different before and after their incorporation into a composition, because, as described below, the particles may consist of polymer comprising a monomer which is soluble or which swells in alkaline media, such that the particles of polymer may swell in water or alkaline media after incorporation into the composition for topical application.

The size of the particles is measured by means of the Brookhaven 90Plus particle size analyser. The measurements are carried out at 90°.

The composition of the invention exhibits both a good foaming power and a good iridescent effect. It exhibits the advantage of having this iridescent effect regardless of the value of its conductance (expressed as ohm⁻¹ or as Siemens)

Macroscopically, the iridescent effect results in variations in colour which are perceived by an observer who would be moving around the surface of the product illuminated by a fixed light beam. By contrast, a product is not iridescent when the observer does not see a change in colour during their movement.

The iridescent effect may be measured by means of a goniometer, the principle of which is to measure the colour by varying the geometric conditions of observations (angles of illumination and of detection of the reflected light). The method of measurement used here and described in greater detail below is inspired by that described in <<Color effects from thin film designs>> by Roger Phillips, Mike Nofi and Robert Slusser (Flex Products Inc 2793 Nothpoint Parkway Santa Rosa Calif. 95407).

### Method of Measurement

The samples, at the temperature of 23° C., fill a vessel 5 mm in height. Their top surface is placed in the plane of measurement of a spectrophotogoniometer. The spectra are measured as a reflexion at 400-760 nm in steps of 5 nm. The spectra are recorded at an angle of illumination set at 55° and at angles of detection placed successively at the following values: 90° (scattering), 100, 110, 120, 130 and 140°. This path simulates the variations perceived by an observer who would be moving around the surface of the product illuminated by a fixed light beam.

The different spectra thus obtained are processed in order to obtain the colorimetric parameters L*a*b* in the CIELAB space (reference: ceramic bore MINOLTA Number 20231050 at the illumination D65 Y=94.1, x=0.3157, y=0.3331). The colour path is obtained by joining in the plane of abscissa a* and of ordinate b*, the coordinates of the colorimetric values calculated. a* varies from green to red and b* varies from blue to yellow. When a* is negative, the colour possesses a dominant green; when it is positive, the colour possesses a dominant red. When b* is negative, the colour possesses a dominant blue; when it is positive, the colour possesses a dominant yellow. The colorimetric difference is equal to the square root of the sum of the squares of the differences in the values a and b relative to the first measurement (detection angle of 90 degrees, a measurement close to that obtained with a conventional calorimeter such as the chromameter MINOLTA CR300).

Thus, a composition has an iridescent appearance when the colorimetric difference for this composition or for the dispersion of particles of polymer which it contains is greater than 2, and preferably varies from 2 to 100, preferably from 3 to 60 for an angle of illumination of 55° and an angle of detection of between 100 and 140° as described above. The incident light is characterized by a beam having a diameter of 8 mm, an angular resolution of 1.3 degrees, a wavelength of 250 to 800 nm and a 1 nm type resolution.

A product which does not possess an iridescent effect is characterized, for example, by differences varying from 0.1 to 1 under the same measurement conditions.

The composition of the invention is also characterized by its turbidity, that is to say its opacity. The turbidity measures the opacity of a product. The NTU (Nephelometric Turbidity Units) are the units of measurement of the turbidity of a composition. The measurement of turbidity may be carried out, for example, with a turbidimeter model 2100P from the company HACH, the tubes used for the measurement being identified by the references AR397A cat 24347-06. The measurements are carried out at room temperature (20° C. to 25° C.). The higher the turbidity, the greater the opacity of the product. The composition of the invention is generally translucent to opaque and it preferably exhibits a turbidity of greater than 100.

The composition according to the invention generally has a viscosity ranging from 1 poise to 35 poises (0.1 Pa.s to 3.5 Pa.s), preferably from 4 to 20 poises (0.4 to 2 Pa.s) and more preferably from 5 to 11 poises (0.5 to 1.1 Pa.s), this viscosity being measured at about 25° C. with a Rheomat 180 apparatus using a 3, 4 or 5 rotor depending on the viscosity range, at 200 s⁻¹.

The composition of the invention is a composition for topical, and in particular cosmetic, use, and, as such, it contains a physiologically acceptable medium, that is to say a medium compatible with the skin, the hair, the nails and/or the mucous membranes (lips). In addition, it preferably has a pH compatible with the skin, that is to say preferably ranging from 3 to 8 and, even better, from 5 to 7. This pH value may be adjusted depending on the polymer dispersions used.

The polymer particles used in the composition of the invention should be monodisperse, that is to say that the results of measurements of the diameter of the particles are statistically distributed around a mean and according to a single Gaussian curve. The variation relative to the mean should not exceed 10% per 100% of particles. This means that the particles practically all have the same size. This size ranges from 50 to 300 nm, preferably from 90 to 230 nm and even better from 100 to 200 nm. The size of the particles is measured by means of the Brookhaven 90Plus particle size analyser. The measurements are carried out at 90°.

The particles consist of polymers. They become suspended or dispersed in water, in latex or emulsion form. Latexes are aqueous dispersions of particles of polymers, as described in <<An introduction to polymer colloids>> by F. CANDAU and R. H. OTTEWILL, Kluwer Academic Publishers, March 1989. The term polymer is understood here to mean both homopolymers obtained from a single type of monomer and copolymers obtained from several types (two or more) of monomers. These polymers may be either associative polymers (that is to say possessing a hydrophobic part and a hydrophilic part) or nonassociative polymers (that is to say hydrophilic or water-soluble). They are dispersible in water and may exhibit swelling in alkaline media (or alkaline-swelling) or not. The polymers may be of any type: nonionic, anionic, cationic, zwitterionic or amphoteric.

The particles which can be used in the composition of the invention preferably consist of ionic polymers and even better of anionic polymers. These polymers are dispersible in water and preferably exhibit alkaline-swelling. The preferred polymers possess at least one monomer soluble in alkaline media, such as monomers of acrylic, methacrylic, vinylacetic, maleic, crotonic and itaconic acids. They may contain another monomer such as butadiene, ethylene, acrylonitrile, chloroprene, vinylidene chloride, isoprene, vinyl chloride, and esters of acrylic, methacrylic or vinylacetic, maleic, crotonic and itaconic acids.

Thus, the polymer particles may be chosen, for example, from the particles consisting of the following anionic polymers:

1) homopolymers of acrylic acid, such as those of the dispersions (in aqueous emulsion) marketed under the names MIRACARE XC 96/36, MIRACARE XC 96/37 and MIRACARE XC 96/52 (acrylic nanolatex) by the company RHODIA CHIMIE.

2) copolymers of acrylic acid and of other monomers, such as those of the dispersions marketed under the names NEOCRYL PD-723-B (particle size: 114 nm), NEOCRYL XK 90 (particle size: 109 nm), NEOCRYL XK 53 (particle size: 96 nm) by the company AVECIA RESINS; ECOCRYL VS 301 (particle size: 150 nm) by the company ATOCHEM; NEOCRYL XK-75 (particle size: 102 nm) (aqueous emulsion of an acrylic acid/butyl methacrylate/methyl methacrylate/butyl acrylate copolymer) by the company AVECIA RESINS; MIRACARE XC 97-8 to 13 (acrylic/methyl methacrylate/butyl acrylate/methacrylic acid copolymer and derivatives in nonionic aqueous nanoemulsion) by the company RHODIA CHIMIE, ACRYSOL 33 (or ACULYN 33) (acrylic acid/ethyl acrylate copolymer) (particle size: 110 nm) by the company ROHM & HAAS.

3) styrene derivatives, such as those of the dispersions marketed under the name SETALUX 6801 AQ 24 (styrene/butyl acrylate/methyl methacrylate/methacrylic acid hybrid copolymer) (particle size: 186 nm) by the company AKZO NOBEL; under the name RHODOPAS GS 125 (particle size: 170 nm) by the company RHONE POULENC, under the names NEOCRYL XK 61 (anionic acrylic/styrene copolymer) (particle size: 76 nm), NEOCRYL A 1079 (styrene/acrylate copolymer) (particle size: 115 nm) by the company AVECIA RESINS; under the name RK-3-59A-PMN (mixture of two styrene/acrylic polymers (nucleus/envelope)) (particle size: 116 nm) by the company ROHM-HAAS, under the name DOW LATEX 432 (styrene/acrylate copolymer) (particle size: 145 nm) by the company LAMBERT-RIVIERE, under the name SCX 8060 (particle size: 116 nm) by the company JOHNSON POLYMER France.

4) fluorinated derivatives, such as those of the dispersions marketed under the references LUMIFLON E-3029 (anionic aqueous dispersion containing 50% of hydroxylated fluorinated polymer) (particle size: 100 nm) and LUMIFLON FE-3000 (anionic aqueous dispersion containing 50% of hydroxylated fluorinated polymer (particle size: 150 nm) by the company ASAHI GLASS.

5) silicone-based derivatives, such as that of the dispersion marketed under the reference HYCAR 26348 (acrylates/acrylonitrogen/siloxane copolymer) (particle size: 133 nm) by the company GOODRICH.

6) diisocyanate derivatives, such as that of the dispersion marketed under the reference NEOPAC E-106 (urethane/acrylate copolymer (and) methylpyrrolidone (and) triethylamine) (particle size: 98 nm) by the company AVECIA RESINS.

According to a preferred embodiment of the invention, particles of acrylic acid/ethyl acrylate copolymer, particles of styrene/butyl acrylate/methyl methacrylate/methacrylic acid hybrid copolymer, that is to say the dispersions sold under the names ACRYSOL 33 or ACULYN 33 by the company ROHM & HAAS and SETALUX 6801 AQ 24 by the company AKZO NOBEL, and mixtures thereof are used. These dispersions are characterized by their colorimetric difference. When the polymer dispersion <<ACULYN 33>> is used, it is preferable that the composition has a pH value ranging from 3 to 6.3.

| | | Size of the | |
|---|---|---|---|
| | Appearance of | particles in | Maximum |
| | the raw | the | colorimetric |
| Raw material | material | dispersion | difference |
| ACULYN 33 | Iridescent | 110.2 ± 2.4% | 12.5 |
| | white milk | | |
| SETALUX 6801 | Iridescent | 187.7 ± 1.8% | 11.1 |
| AQ 24 | white milk | | |
| Comparative | Non- | 252.2 ± 13.6% | Less than 1 |
| example: | iridescent | With two | |
| SYNTHALEN | white milk | populations | |
| W2000 ® of | | of sizes, one | |
| 3V SA | | centered | |
| | | around 167 nm | |
| | | and the other | |
| | | around 370 nm | |

As shown in the table above, the particles of SYNTHALEN W2000 are not monodisperse, that is to say that their size is not homogeneous and comprises several Gaussian curves. The comparative results presented later show that this polymer dispersion is not appropriate for obtaining an iridescent effect, whereas the two dispersions <<ACULYN 33>> and <<SETALUX 6801 AQ 24>> which are monodisperse allow iridescent compositions to be obtained.

The quantity of polymer particles in dispersion, in the composition of the invention, depends on the polymer used. It is preferably at least 3% by weight of active material and it may range, for example, from 3 to 50%, preferably from 3.5 to 40% and even better from 5 to 30% by weight of active material (that is to say by weight of dry matter of particles of polymers) relative to the total weight of the composition.

The composition according to the invention contains at least one alkyl polyglycoside (abbreviated APG). The total quantity of alkyl polyglycoside(s) may range, for example, by weight of active material, from 0.5 to 50% by weight and even better from 1 to 30% by weight of active material relative to the total weight of the composition.

The alkyl polyglycosides which may be used in the composition of the invention may be more particularly represented by the following general formula (I):

R-O-(G)x (I)

in which R represents a saturated or unsaturated, linear or branched alkyl radical comprising from 6 to 30 carbon atoms, G represents a reduced sugar comprising from 5 to 6 carbon atoms, and x denotes a value ranging from 1 to 15.

Alkyl polyglycosides preferred according to the present invention are compounds of formula (I) in which R denotes more particularly an alkyl radical comprising from 6 to 30 carbon atoms and preferably from 8 to 16 carbon atoms, G denotes glucose, fructose or galactose, x is a value ranging from 1 to 4 and more particularly from 1 to 3. According to a preferred embodiment of the invention, the alkyl polyglycoside used in the composition of the invention is an alkyl polyglucoside, that is to say a compound of formula (I) in which G denotes glucose and x is a value ranging from 1.2 to 3.

As alkyl polyglucosides, there may be mentioned, for example, decyl glucoside (C9/C11 alkyl polyglucoside (1.4)) such as the product marketed under the name MYDOL 10 by the company Kao Chemicals, the product marketed under the name PLANTAREN 2000 UP and PLANTACARE 2000 UP by the company Henkel, and the product marketed under the name ORAMIX NS 10 by the company Seppic; caprylyl/capryl glucoside such as the product marketed under the name ORAMIX CG 110 by the company Seppic or under the name LUTENSOL GD 70 by the company BASF; lauryl glucoside such as the products marketed under the names PLANTAREN 1200 N and PLANTACARE 1200 by the company Henkel; and coco-glucoside such as the product marketed under the name PLANTACARE 818/UP by the company Henkel, and mixtures thereof.

The alkyl polyglycosides may be optionally combined with one or more other surfactants chosen from nonionic, anionic, amphoteric or zwitterionic surfactants, and mixtures thereof.

1) As nonionic surfactants, there may be mentioned, for example, esters of polyols and of fatty acids, esters of polyethylene glycols and of fatty acid, derivatives of fatty alcohols and of polyols (ethers), and oxyalkylenated (oxyethylenated and/or oxypropylenated) derivatives of these compounds. There may also be mentioned maltose esters, polyglycerolated fatty alcohols, glucamine derivatives such as 2-ethylhexyl-oxycarbonyl-n-methylglucamine and mixtures thereof.

The maltose derivatives are, for example, those disclosed in document EP-A-566 438, such as O-octanoyl-6′-D-maltose or O-dodecanoyl-6′-D-maltose disclosed in document FR-2 739 556.

Among the polyglycerolated fatty alcohols which may be mentioned are polyglycerolated dodecanediol (3.5 mol of glycerol), this product being sold under the name CHIMEXANE NF by the company Chimex.

2) Anionic surfactants which may be used, for example, include carboxylates, amino acid derivatives, alkyl sulphates, alkyl ether sulphates, sulphonates, isethionates, taurates, sulphosuccinates, alkyl sulphoacetates, phosphates and alkyl phosphates, polypeptides, anionic alkyl polyglucoside derivatives and fatty acid soaps, and mixtures thereof.

Carboxylates which may be mentioned, for example, include alkali metal salts of N-acylamino acids; amido ether carboxylates (AECs), for instance sodium lauryl amido ether carboxylate (3 EO) sold under the name AKYPO FOAM 30 by the company Kao Chemicals; polyoxyethylenated carboxylic acid salts, for instance oxyethylenated (6 EO) sodium lauryl ether carboxylate (C12-14-16 65/25/10) sold under the name AKYPO SOFT 45 NV by the company Kao Chemicals; polyoxyethylenated fatty acids of olive oil and of carboxymethyl, for instance the product being sold under the name OLIVEM 400 by the company Biologia E Tecnologia; oxyethylenated (6 EO) sodium tridecyl ether carboxylate sold under the name NIKKOL ECTD-6NEX by the company NIKKOL and sodium 2-(2-hydroxy-alkyloxy)acetate sold under the name BEAULIGHT SHAA by the company SANYO.

The amino acid derivatives may be chosen, for example, from sarcosinates and in particular acylsarcosinates, for instance the sodium lauroyl sarcosinate sold under the name SARKOSYL NL 97 by the company Ciba or sold under the name ORAMIX L 30 by the company Seppic, the sodium myristoyl sarcosinate sold under the name NIKKOL SARCOSINATE MN by the company Nikkol or the sodium palmitoyl sarcosinate sold under the name NIKKOL SARCOSINATE PN by the company Nikkol; alaninates, for instance the sodium N-lauroyl-N-methylamidopropionate sold under the name SODIUM NIKKOL ALANINATE LN 30 by the company Nikkol or sold under the name ALANONE ALE by the company Kawaken, and the N-lauroyl-N-methylalanine triethanolamine sold under the name ALANONE ALTA by the company Kawaken; N-acylglutamates, for instance the triethanolamine monococoylglutamate sold under the name ACYLGLUTAMATE CT-12 by the company Ajinomoto and the triethanolamine lauroylglutamate sold under the name ACYLGLUTAMATE LT-12 by the company Ajinomoto; aspartates, for instance the mixture of triethanolamine N-lauroyl aspartate and of triethanolamine N-myristoylaspartate, sold under the name ASPARACK LM-TS2 by the company Mitsubishi; citrates, and mixtures thereof.

Glycine derivatives which may be mentioned include sodium N-cocoylglycinate and potassium N-cocoylglycinate, for instance the products sold under the names AMILITE GCS-12 and AMILITE GCK-12 by the company Ajinomoto.

Alkyl ether sulphates which may be mentioned, for example, include the sodium lauryl ether sulphate (C12-14 70/30) (2.2 EO) sold under the names SIPON AOS 225 or TEXAPON N702 PATE by the company Henkel, the ammonium lauryl ether sulphate (C12-14 70/30) (3 EO) sold under the name SIPON LEA 370 by the company Henkel, and the ammonium (C12-C14) alkyl ether (9 EO) sulphate sold under the name RHODAPEX AB/20 by the company Rhodia Chimie.

Sulphonates which may be mentioned, for example, include alpha-olefin sulphonates, for instance the sodium alpha-olefin sulphonate (C14-16) sold under the name BIO-TERGE AS-40 by the company Stepan, sold under the names WITCONATE AOS PROTEGE and SULFRAMINE AOS PH 12 by the company Witco or sold, under the name BIO-TERGE AS-40 CG by the company Stepan, the sodium secondary olefin sulphonate sold under the name HOSTAPUR SAS 30 by the company Clariant; linear alkyl aryl sulphonates, for instance the sodium xylene sulphonate sold under the names MANROSOL SXS30, MANROSOL SXS40 and MANROSOL SXS93 by the company Manro.

Isethionates which may be mentioned include acylisethionates, for instance sodium cocoylisethionate, such as the product sold under the name JORDAPON CI P by the company Jordan.

Taurates which may be mentioned include the sodium salt of palm kernel oil methyltaurate sold under the name HOSTAPON CT PATE by the company Clariant; N-acyl N-methyltaurates, for instance the sodium N-cocoyl N-methyltaurate sold under the name HOSTAPON LT-SF by the company Clariant or sold under the name NIKKOL CMT-30-T by the company Nikkol, and the sodium palmitoyl methyltaurate sold under the name NIKKOL PMT by the company Nikkol.

Sulphosuccinates which may be mentioned, for example, include the oxyethylenated (3 EO) lauryl alcohol monosulphosuccinate (C12/C14 70/30) sold under the names SETACIN 103 SPECIAL, REWOPOL SB-FA 30 K 4 by the company Witco, the disodium salt of a C12-C14 alkyl hemisulphosuccinate, sold under the name SETACIN F SPECIAL PASTE by the company Zschimmer Schwarz, the oxyethylenated (2 EO) disodium oleamidosulphosuccinate sold under the name STANDAPOL SH 135 by the company Henkel, the oxyethylenated (5 EO) laurylamide monosulphosuccinate sold under the name LEBON A-5000 by the company Sanyo, the oxyethylenated (10 EO) disodium salt of lauryl citrate monosulphosuccinate sold under the name REWOPOL SB CS 50 by the company Witco, and the ricinoleic monoethanolamide monosulphosuccinate sold under the name REWODERM S 1333 by the company Witco.

Phosphates and alkyl phosphates which may be mentioned, for example, include monoalkyl phosphates and dialkyl phosphates, such as the lauryl monophosphate sold under the name MAP 20 by the company Kao Chemicals, the potassium salt of dodecylphosphoric acid, as a mixture of monoester and diester (mainly diester), sold under the name CRAFOL AP-31 by the company Cognis, the mixture of monoester and diester of octylphosphoric acid, sold under the name CRAFOL AP-20 by the company Cognis, the mixture of ethoxylated (7 mol of EO) phosphoric acid monoester and diester of 2-butyloctanol, sold under the name ISOFOL 12 7 EO-PHOSPHATE ESTER by the company Condea, the potassium salt or triethanolamine salt of monoalkyl (C12-C13) phosphate sold under the references ARLATONE MAP 230K-40 and ARLATONE MAP 230T-60 by the company Uniqema, and the potassium lauryl phosphate sold under the name DERMALCARE MAP XC-99/09 by the company Rhodia Chimie.

The polypeptides are obtained, for example, by condensing a fatty chain with amino acids from cereals and in particular from wheat and oat. Polypeptides which may be mentioned, for example, include the potassium salt of hydrolysed lauroyl wheat protein, sold under the name AMINOFOAM W OR by the company Croda, the triethanolamine salt of hydrolysed cocoyl soybean protein, sold under the name MAY-TEIN SY by the company Maybrook, the sodium salt of oat lauroyl amino acids, sold under the name PROTEOL OAT by the company SEPPIC, the collagen hydrolysate grafted onto coconut fatty acid, sold under the name GELIDERM 3000 by the company Deutsche Gelatine, and the soybean proteins acylated with hydrogenated coconut acids, sold under the name PROTEOL VS 22 by the company Seppic.

The anionic derivatives of alkyl polyglucosides may be, in particular, glyceryl ethers, carbonates, sulphosuccinates, tartrates and citrates obtained from alkyl polyglucosides. Mention may be made, for example, of the sodium salt of cocoylpolyglucoside (1,4) tartaric ester, sold under the name EUCAROL AGE-ET by the company Cesalpinia, the disodium salt of cocoylpolyglucoside (1,4) sulphosuccinic ester, sold under the name ESSAI 512 MP by the company Seppic, and the sodium salt of cocoylpolyglucoside (1,4) citric ester, sold under the name EUCAROL AGE-EC® by the company Cesalpinia. Another anionic saccharide derivative may be sodium dodecyl-D-galactoside uronate sold under the name DODECYL-D-GALACTOSIDE URONATE DE SODIUM by the company SOLIANCE.

The fatty acid soaps which may be used as anionic surfactants are fatty acids of natural or synthetic origin, salified with a mineral or organic base. The fatty chain may comprise from 6 to 22 carbon atoms and preferably from 8 to 18 carbon atoms. The mineral or organic base may be chosen from alkali metals or alkaline-earth metals, amino acids and amino alcohols. Salts which may be used, for example, include the sodium, potassium, magnesium, triethanolamine and N-methylglucamine salts of lysine and of arginine. Soaps which may be mentioned, for example, include the potassium or sodium salts of lauric, myristic, palmitic or stearic acid (potassium or sodium laurate, myristate, palmitate and stearate), and mixtures thereof.

3) The amphoteric and zwitterionic surfactants which may be mentioned, for example, include betaines, N-alkylamidobetaines and derivatives thereof, glycine derivatives, sultaines, alkyl polyaminocarboxylates and alkylamphoacetates, and mixtures thereof.

Betaines which may be mentioned, for example, include cocobetaine, for instance the product sold under the name DEHYTON AB-30 by the company Henkel, laurylbetaine, for instance the product sold under the name GENAGEN KB by the company Clariant, oxyethylenated (10 EO) laurylbetaine, for instance the product sold under the name LAURYL ETHER (10 EO) BETAINE by the company Shin Nihon Rica, and oxyethylenated (10 EO) stearylbetaine, for instance the product sold under the name STEARYL ETHER (10 EO) BETAINE by the company Shin Nihon Rica.

Among the N-alkylamidobetaines and derivatives thereof which may be mentioned, for example, are the cocamidopropylbetaine sold under the name LEBON 2000 HG by the company Sanyo, or sold under the name EMPIGEN BB by the company Albright & Wilson, and the lauramidopropyl betaine sold under the name REWOTERIC AMB12P by the company Witco.

Sultaines which may be mentioned include the cocoylamidopropylhydroxysulphobetaine sold under the name CROSULTAINE C-50 by the company Croda.

Alkyl polyaminocarboxylates (APACs) which may be mentioned include the sodium cocoylpolyaminocarboxylate sold under the name AMPHOLAK 7 CX/C and AMPHOLAK 7 CX by the company Akzo Nobel, the sodium stearylpolyamidocarboxylate sold under the name AMPHOLAK 7 TX/C by the company Akzo Nobel and the sodium carboxymethyloleylpolypropylamine sold under the name AMPHOLAK X07/C by the company Akzo Nobel.

Alkylamphoacetates which may be mentioned, for example, include N-disodium N-cocoyl-N-carboxymethoxyethyl-N-carboxymethylethylenediamine (CTFA name: disodium cocamphodiacetate), for instance the product sold under the name MIRANOL C2M CONCENTRE NP by the company Rhodia Chimie and N-sodium N-cocoyl-N-hydroxyethyl-N-carboxymethylethylenediamine (CTFA name: sodium cocamphoacetate).

According to one particular embodiment of the invention, among the surfactants other than the APGs mentioned above, the surfactants more particularly used in the composition of the invention are (1) from among the anionic surfactants: acylsarcosinates, oxyethylenated alkyl ether sulphates, N-acyl N-methyltaurates, N-acylglutamates, N-acylisethionates, N-acylglycinates, sulphosuccinates, phosphates and alkyl phosphates, acyl-D-galactoside uronate, sodium 2-( 2-hydroxyalkyloxy)acetate, polypeptides and soaps; (2) from among the amphoteric and zwitterionic surfactants: betaines and alkyl amphoacetates; (3) from among the nonionic surfactants: O-octanoyl-6′-D-maltose, O-dodecanoyl-6′-D-maltose, polyglycerolated dodecanediol (3.5 mol of glycerol) and 2-ethylhexyloxycarbonyl-N-methylglucamine; and mixtures of these surfactants.

The total quantity of surfactants (APG +other surfactants) may range, for example, by weight of active material, from 2 to 50% by weight and even better from 3 to 30% by weight relative to the total weight of the composition.

It is preferable that, when the composition comprises one or more surfactants other than alkyl polyglycosides, the quantity of alkyl polyglycoside(s) represents at least 60% by weight of active material relative to the total quantity of surfactants (APG +other surfactants).

The physiologically acceptable medium of the composition of the invention comprises water. The quantity of water may range from 30 to 96.5%, preferably 40 to 95% by weight relative to the total weight of the composition. It may contain, in addition to water, one or more solvents chosen from lower alcohols comprising from 1 to 6 carbon atoms, such as ethanol; polyols such as glycerin; glycols such as butylene glycol, isoprene glycol, propylene glycol, polyethylene glycols such as PEG-8; sorbitol; sugars such as glucose, fructose, maltose, lactose, sucrose; and mixtures thereof. The quantity of solvent(s) in the composition of the invention may range from 0.5 to 30% by weight and preferably from 5 to 20% by weight relative to the total weight of the composition.

The composition of the invention may also contain customary additives in the cosmetic field, such as mattifying inorganic or organic fillers, active agents, preservatives, gelling agents, plasticizers, antioxidants, perfumes, odour absorbers, sequestrants (EDTA), acidic or basic pH regulators, or buffers, pigments and pearlescent agents (in a quantity such that this does not disrupt the desired effect), nonionic, anionic, cationic and amphoteric polymers, fatty substances made compatible with the aqueous medium, such as oils or waxes, in so far as the additive does not impair the properties sought for the composition of the invention. The quantities of these various additives are those conventionally used in the fields considered, and for example from 0.01 to 20% of the total weight of the composition.

As active agents, it is possible to incorporate any active agent normally used in the cosmetic and dermatological fields, such as water-soluble or fat-soluble vitamins or provitamins, for example vitamins A (retinol), C (ascorbic acid), B3 or PP (niacinamide), B5 (panthenol), E (tocopherol), K1, beta-carotene, and the derivatives of these vitamins and in particular their esters; hormones or derivatives such as DHEA and 7α-hydroxy DHEA; antiseptics; antiseborrhoeics; antimicrobials such as benzoyl peroxide, salicylic acid, triclosan, azelaic acid, niacinamide (vit. PP); moisturizers such as glycerin, hyaluronic acid, pyrrolidone carboxylic acid (PCA) and its salts, sodium pidolate, serine, xylitol, trehalose, ectoine, ceramides, urea; keratolytic and antiageing agents such as alpha-hydroxy acids such as glycolic acid, citric acid, lactic acid, beta-hydroxy acids such as salicylic acid, coenzyme Q10; sunscreens; optical brighteners; slimming agents such as caffeine, theophylline, theobromine. It is also possible to use a mixture of two or more of these active agents. The active agent(s) may be, for example, present in a concentration ranging from 0.01 to 20%, preferably from 0.1 to 10% and even better from 0.5 to 5% of the total weight of the composition.

The composition of the invention may be provided in any of the galenic forms normally used in the cosmetic and dermatological fields. It is generally provided in the form of a gel and is prepared according to the customary methods, that is to say by mixing the polymer and the surfactants in the aqueous medium.

The composition according to the invention may comprise an oily phase (or fatty phase) comprising at least one oil, provided that it is solubilized in the medium, for example by forming a microemulsion. The oil(s) may be chosen from oils of plant origin (jojoba, avocado, sesame, sunflower, maize, soyabean, safflower, grapeseed), mineral oils (petroleum jelly, hydrogenated or nonhydrogenated isoparaffins, isohexadecane, squalane), synthetic oils (parleam, isopropyl myristate, cetearyl octanoate, polyisobutylene, ethylhexyl palmitate, alkyl benzoates), volatile or nonvolatile silicone oils such as polydimethylsiloxanes (PDMS) and cyclodimethylsiloxanes or cyclomethicones, and fluorinated or fluorosilicone oils, as well as mixtures of these oils. The oily phase may contain, in addition, other fatty constituents such as fatty alcohols such as stearyl alcohol, cetyl alcohol and the mixture thereof (cetearyl alcohol); fatty acids; waxes; silicone gums. The oily phase may be present in a quantity ranging from 0.01 to 20% by weight relative to the total weight of the composition.

As indicated above, the composition of the invention may advantageously constitute a foaming composition, in particular for removing make-up from and/or cleansing the skin, including the scalp, the hair, the lips or the mucous membranes. This composition not only exhibits an iridescent colour, but also an excellent foaming power.

Accordingly, the subject of the present invention is the cosmetic use of a composition as defined above, for removing make-up from and/or cleansing the skin, the lips and/or the hair.

The compositions according to the invention may be useful as cleansing compositions, for example, in the following two ways:

the first use consists in spreading the gel in the hands, in applying it to the face or to the body and then in massaging it in the presence of water in order to develop the foam directly on the face or the body.

the other possible use of this type of product consists in developing the foam in the palms of the hands before being applied to the face or the body.

In both cases, the foam is then rinsed off.

Another subject of the invention consists of a cosmetic method of cleansing the skin, the scalp and/or the hair, characterized in that the composition of the invention is applied to the skin, to the scalp and/or to the hair, in the presence of water, and in that the foam formed and the residues of dirt are removed by rinsing with water.

To evaluate the foaming power of the compositions of the invention, use is made of their sensory qualities. The sensory qualities of the compositions obtained are evaluated according to the following protocol:

Before any use of the products, the hands are washed with Marseilles soap, then appropriately rinsed and dried, and then the following protocol is followed:

1- wet the hands by passing them under running water, shake them three times to drain them;

2- place 1 g of product in the hollow of one of the hands;

3- work the product between both palms for 10 seconds;

4- add 2 ml of water and work the product again for 10 seconds;

5- rinse the hands under water;

6- wipe them.

The criteria are evaluated at each stage of the protocol and are noted on a scale from 0 to 10:

step 3: evaluation of the covering power: the higher the mark awarded, the more the skin cannot be seen through the product spread;

step 4: evaluation of the foam quality:

The volume of foam: the higher the mark awarded, the larger the volume;

The size of the bubbles constituting the foam: the higher the mark awarded, the larger the bubbles;

The density: consistency, retention of the foam; the higher the mark awarded, the higher the density;

The smoothness of the foam: the higher the mark awarded, the smoother the foam;

step 5: evaluation during rinsing:

The rinsing: the lower the mark awarded, the greater the presence of a slippery film which is difficult to remove.

The following examples illustrate the invention. A.M. means therein <<as active material>>. The quantities are indicated as a percentage by weight unless otherwise stated.

### EXAMPLE 1:

### Foaming Composition for Cleansing the Face

| | | |
|---|---|---|
| ACUYLN 33 ® from ROHM-HAAS | 7.5% | A.M. |
| Decyl glucoside (Plantacare 2000 UP | 6% | A.M. |
| from COGNIS, containing 55% of A.M. | | |
| in water) | | |
| Glycerin | 3% | |

| | |
|---|---|
| Preservatives | qs |
| Sodium hydroxide at 10% | qs pH |
| Water | qs 100% |

Procedure: All the constituents, except ACULYN 33 and sodium hydroxide, are solubilized in water. ACUYLN 33. is added, the mixture is homogenized and the pH is adjusted with sodium hydroxide.

An iridescent translucent gel of pH 5.85 is obtained whose colours vary from blue, green, yellow, to orange depending on the orientation. This gel has a viscosity, after 10 minutes, measured with rotor 3 or 4 (Rhéomat RM180 at room temperature, that is to say at about 25° C., at 200 s⁻¹), of 8.2 poises (0.82 Pa.s), a colorimetric difference of 53.9 (for an angle of illumination of 55° and an angle of reception of 120°) and it has a conductance, measured by means of the CD78 type conductimeter from TACUSSEL at 25° C., of 4283 μΩ⁻¹.

Furthermore, this gel has good foaming properties.

### EXAMPLE 2

### Foaming Composition for Cleansing the Face

| | | |
|---|---|---|
| ACUYLN 33 ® from ROHM-HAAS | 7.5% | A.M. |
| Decyl glucoside (Plantacare 2000 UP | 6% | A.M. |
| from COGNIS, containing 55% of A.M. | | |
| in water) | | |
| Glycerin | 3% | |
| Sodium chloride | 1% | |

| | |
|---|---|
| Preservatives | qs |
| Sodium hydroxide at 10% | qs pH |
| Water | qs 100% |

The procedure is identical to that of Example 1.

An iridescent translucent gel of pH 5.86 is obtained whose colours vary from blue, green, yellow, to orange depending on the orientation. This gel has a viscosity, after 10 minutes, measured with rotor 3 or 4 (Rhéomat RM180 at room temperature, that is to say at about 25° C., at 200 s⁻¹), of 3 poises (0.3 Pa.s), and it has a conductance, measured by means of the CD78 type conductimeter from TACUSSEL at 25° C., of 14406 μΩ⁻¹.

This example shows that the addition of salt (sodium chloride) does not affect the iridescent character of the composition.

Furthermore, this gel has good foaming properties.

### EXAMPLE 3

### Foaming Composition for Cleansing the Face

| | | |
|---|---|---|
| SETALUX 6801 AQ64 (AKZO NOBEL) | 18% | A.M. |
| Decyl glucoside (Plantacare 2000 UP | 6% | A.M. |
| from COGNIS, containing 55% of A.M. | | |
| in water) | | |
| Glycerin | 3% | |

| | |
|---|---|
| Preservatives | qs |
| Sodium hydroxide at 10% | qs pH |
| Water | qs 100% |

The procedure is identical to that of Example 1.

An iridescent translucent gel of pH 6.8 is obtained whose colours vary from blue, green, yellow, to orange depending on the orientation. This gel has a viscosity, after 10 minutes, measured with rotor 3 or 4 (Rhéomat RM180 at room temperature, that is to say at about 25° C., at 200 s⁻¹), of 12 poises (1.2 Pa.s). Furthermore, this gel has good foaming properties.

### EXAMPLE 4

### Foaming Composition for Cleansing the Body Shower Gel

| | | |
|---|---|---|
| ACULYN 33 ® from ROHM-HAAS | 5% | A.M. |
| Decyl glucoside (Plantacare 2000 UP | 13% | A.M. |
| from COGNIS, containing 55% of A.M. | | |
| in water) | | |
| Glycerin | 3% | |

| | |
|---|---|
| Preservatives | qs |
| Sodium hydroxide at 10% | qs pH |
| Water | qs 100% |

The procedure is identical to that of Example 1.

An iridescent translucent gel of pH 5.86 is obtained whose colours vary from blue, green, yellow, to orange depending on the orientation. This gel has a viscosity, after 10 minutes, measured with rotor 3 or 4 (Rhéomat RM180 at room temperature, that is to say at about 25° C,, at 200 s⁻¹), of 11 poises (1.1 Pa.s).

The sensory qualities of this gel are very good. Thus, in the foaming test described above, the results obtained are the following:

| | |
|---|---|
| Speed of appearance of the first bubbles: | 8.1 |
| Covering power | 6.3 |
| Volume of the foam | 6.6 |
| Size of the bubbles | 4.3 |
| Density | 7.7 |
| Smoothness of the foam | 7.7 |
| Rinsing | 9.1 |

It is observed that the foam qualities are good: rapid appearance of bubbles, good volume and good density of the foam, small size of the bubbles. Furthermore, the rinsing is carried out very easily.

### EXAMPLE 5

### Foaming Composition for Cleansing the Body Shower Gel

| | | |
|---|---|---|
| ACULYN 33 ® from ROHM-HAAS | 5% | A.M. |
| Decyl glucoside (Plantacare 2000 UP | 11% | A.M. |
| from COGNIS, containing 55% of A.M. | | |
| in water) | | |
| Sodium lauryl ether sulphate (TEXAPON | 2% | A.M. |
| N702 PATE from HENKEL, at 70% of A.M. | | |
| in water) | | |
| Glycerin | 3% | |

| | |
|---|---|
| Preservatives | qs |
| Sodium hydroxide at 10% | qs pH |
| Water | qs 100% |

The procedure is identical to that of Example 1.

An iridescent translucent gel of pH 5.8 is obtained whose colours vary from blue, green, yellow, to orange and which has a viscosity, after 10 minutes, measured with rotor 3 or 4 (Rhéomat RM180 at room temperature, that is to say at about 25° C., at 200 s^{31 1}), of 11 poises (1.1 Pa.s).

The sensory qualities of this gel are very good. Thus, in the foaming test described above, the results obtained are the following:

| | |
|---|---|
| Speed of appearance of the first bubbles: | 9.5 |
| Covering power | 6.5 |
| Volume of the foam | 6.3 |
| Size of the bubbles | 3.9 |
| Density | 7.3 |
| Smoothness of the foam | 7.9 |
| Rinsing | 9.0 |

It is observed that the foam qualities are good despite the presence of sodium lauryl ether sulphate: rapid appearance of bubbles, good density of foam and great ease of rinsing.

### EXAMPLE 6

### Foaming Composition for Cleansing the Face and the Body

| | | |
|---|---|---|
| SETALUX 6801 AQ64 (AKZO NOBEL) | 18% | A.M. |
| Decyl glucoside (Plantacare 2000 UP | 6% | A.M. |
| from COGNIS, containing 55% of A.M. | | |
| in water) | | |
| Glycerin | 3% | |

| | |
|---|---|
| Preservatives | qs |
| Sodium hydroxide at 10% | qs pH |
| Water | qs 100% |

The procedure is identical to that of Example 1.

An iridescent translucent gel of pH 6.8 is obtained whose colours vary from blue to green depending on the orientation. This gel has a viscosity, after 10 minutes, measured with rotor 3 or 4 (Rhéomat RM180 at room temperature, that is to say at about 25° C., at 200 s⁻¹) , of 3.2 poises (0.32 Pa.s).

The sensory qualities of this gel are very good. Thus, in the foaming test described above, the results obtained are the following:

| | |
|---|---|
| Speed of appearance of the first bubbles: | 9.8 |
| Covering power | 5.2 |
| Volume of the foam | 6.2 |
| Size of the bubbles | 5.3 |
| Density | 7.4 |
| Smoothness of the foam | 5.5 |
| Rinsing | 9.5 |

It is observed that the bubbles appear very rapidly, that the density of the foam is good and that the rinsing is very easy.

### Comparative Example 1

Example 4 is repeated, but replacing Plantacare with an equivalent quantity (13% of A.M.) of a mixture of 6.5% by weight of A.M. of an anionic surfactant, sodium lauryl ether sulphate (TEXAPON N702 PATE marketed by the company HENKEL, at 70% of A.M. in water) and 6.5% by weight of A.M. of an amphoteric surfactant, cocoamidopropylbetaine at 30% of A.M. in water (EMPIGEN BB marketed by the company Albright & Wilson).

An iridescent gel is obtained, but this gel has poor foaming performance features: the covering power, the volume of foam and the size of the bubbles are low. The results of the sensory tests as described above, are indicated below for Example 4 and comparative Example 1:

| | Example 4 | |
|---|---|---|
| | according to the | Comparative |
| Composition | invention | Example 1 |
| Speed of appearance of | 8.2 | 0.0 |
| the first bubbles | | |
| Covering power | 6.3 | 3.2 |
| Volume of the foam | 6.6 | 3.5 |
| Size of the bubbles | 4.3 | 2.0 |
| Density | 7.7 | 5.5 |
| Smoothness of the foam | 7.7 | 8.3 |
| Rinsing | 9.1 | 7.4 |
| Comments | Good volume and | Poor qualities |
| | good smoothness | of the foam |
| | of the foam | |

### Comparative Example 2

ACULYN 33 is replaced, in Example 1, with an equivalent quantity of a dispersion of silica particles: Opalesque 1015 or Opalesque 1030 marketed by the company IKEDA, 15% or 30% dispersions in water of silica particles having a size of about 10 100 nm.

A noniridescent white gel is obtained.

This comparative example shows that only the polymer particles make it possible to obtain the desired result.

1. Iridescent composition for topical application, comprising at least one surfactant chosen from alkyl polyglycosides, and monodisperse particles of polymer in aqueous dispersion, the said particles having a mean size in numerical terms ranging from 50 to 300 nm.

2. Composition according to the preceding claim, characterized in that it exhibits a turbidity of greater than 100.

3. Composition according to either of the preceding claims, characterized in that the said composition and/or the dispersion of particles possess a colorimetric difference ranging from 2 to 100.

4. Composition according to any one of the preceding claims, characterized in that it has a viscosity ranging from 0.1 Pa.s to 3 Pa.s, preferably from 0.4 to 2 Pa.s.

5. Composition according to any one of the preceding claims, characterized in that the particles comprise polymer obtained from one or more monomers chosen from styrene, butadiene, ethylene, acrylonitrile, chloroprene, vinylidene chloride, isoprene, isobutylene and vinyl chloride, acrylic, methacrylic, vinylacetic, maleic, crotonic and itaconic acids and esters thereof, and mixtures thereof.

6. Composition according to any one of the preceding claims, characterized in that the particles comprise anionic polymer.

7. Composition according to the claim 1, characterized in that the particles comprise homopolymers of acrylic acid, copolymers of acrylic acid, styrene derivatives, fluorinated derivatives, silicone-based derivatives, diisocyanate derivatives.

8. Composition according to claim 1, characterized in that the polymer particles are chosen from particles of acrylic acid/ethyl acrylate copolymer, particles of styrene/butyl acrylate/methyl methacrylate/methacrylic acid hybrid copolymer, and mixtures thereof.

9. Composition according to any one of the preceding claims, characterized in that the quantity of polymer particles is at least 3% by weight of active material relative to the total weight of the composition.

10. Composition according to any one of the preceding claims, characterized in that the quantity of polymer particles ranges from 3 to 50% by weight and preferably from 3.5 to 40% by weight of active material relative to the total weight of the composition.

11. Composition according to any one of the preceding claims, characterized in that the quantity of alkyl polyglycoside(s) ranges from 0.5 to 50% by weight of active material and preferably from 1 to 30% by weight of active material relative to the total weight of the composition.

12. Composition according to any one of the preceding claims, characterized in that the alkyl polyglycoside is represented by the following general formula (I):
R-O-(G)ₓ (I)
in which R represents a saturated or unsaturated, linear or branched alkyl radical comprising from 6 to 30 carbon atoms, G represents a reduced sugar comprising from 5 to 6 carbon atoms, and x denotes a value ranging from 1 to 15.

13. Composition according to the preceding claim, characterized in that in formula (I) R denotes an alkyl radical comprising from 6 to 30 carbon atoms and preferably from 8 to 16 carbon atoms, G denotes glucose, fructose or galactose, x is a value ranging from 1 to 4.

14. Composition according to the preceding claim, characterized in that in formula (I) G denotes glucose and x is a value ranging from 1.2 to 3.

15. Composition according to any one of the preceding claims, characterized in that the alkyl polyglycoside is an alkyl polyglucoside chosen from decyl glucoside, caprylyl/capryl glucoside, lauryl glucoside, cocoglucoside and mixtures thereof.

16. Composition according to any one of the preceding claims, characterized in that it comprises, in addition, one or more other surfactants chosen from nonionic, anionic, amphoteric or zwitterionic surfactants, and mixtures thereof.

17. Composition according to the preceding claim, characterized in that the surfactant is chosen from esters of polyols and of fatty acids, esters of polyethylene glycols and of fatty acids, derivatives of fatty alcohol and of polyols, and their oxyalkylenated derivatives; maltose esters; polyglycerolated fatty alcohols; glucamine derivatives; carboxylates; amino acid derivatives; alkyl sulphates; alkyl ether sulphates; sulphonates; isethionates; taurates; sulphosuccinates; alkyl sulphoacetates; phosphates and alkyl phosphates; polypeptides; anionic alkyl polyglucoside derivatives; fatty acid soaps; betaines, N-alkylamidobetaines and derivatives thereof; glycine derivatives; sultaines; alkyl polyaminocarboxylates, alkyl amphoacetates; and mixtures thereof.

18. Composition according to claim 16 or 17, characterized in that the surfactant is chosen from acyl sarcosinates, oxyethylenated alkyl ether sulphates, N-acyl-N-methyltaurates, N-acylglutamates, N-acylisethionates, N-acylglycinates, sulphosuccinates, phosphates and alkyl phosphates, acyl-D-galactoside uronate, sodium 2-(2-hydroxyalkyloxy)acetate, polypeptides, soaps, betaines, alkyl amphoacetates, O-octanoyl-6′-D-maltose, o-dodecanoyl-6′-D-maltose, polyglycerolated dodecanediol (3.5 mol of glycerol), 2-ethylhexyloxycarbonyl-N-methylglucamine; and mixtures thereof.

19. Composition according to any one of claims 16 to 18, characterized in that the total quantity of surfactants ranges from 2 to 50% by weight of active material relative to the total weight of the composition.

20. Composition according to any one of claims 16 to 19, characterized in that the quantity of alkyl polyglycoside(s) represents at least 60% by weight of active material relative to the total quantity of surfactants.

21. Composition according to any one of the preceding claims, characterized in that it comprises 30 to 96.5% by weight of water relative to the total weight of the composition.

22. Composition according to any one of the preceding claims, characterized in that it comprises one or more solvents chosen from alcohols comprising from 1 to 8 carbon atoms, polyols, sugars and mixtures thereof.

23. Composition according to any one of the preceding claims, characterized in that it constitutes a foaming composition.

24. Cosmetic use of the composition according to any one of claims 1 to 22, for removing make-up from and/or cleansing the skin, the lips and/or the hair.

25. Cosmetic method of cleansing the skin, the scalp and/or the hair, characterized in that the composition according to any one of claims 1 to 22 is applied to the skin, to the scalp and/or to the hair, in the presence of water, and in that the foam formed and the residues of dirt are removed by rinsing with water.
